(19) United States
(12) Patent Application Publication
(10) Pub. No.: US 2005/0119263 A1
(43) Pub. Date: Jun. 2, 2005

(54) Treatment of breast cancer

(76) Inventors: Mark David Vincent, London (CA); Lome J. Brandes, Winipeg (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/494,961

(22) PCT Filed: Nov. 6, 2002
(86) PCT No.: PCT/CA02/01725

Related U.S. Application Data
(60) Provisional application No. 60/331,142, filed on Nov. 9, 2001.
(60) Provisional application No. 60/407,241, filed on Sep. 3, 2002.

(51) Int. Cl. A61K 31/537; A61K 31/495; A61K 31/138

(52) U.S. Cl. 514/237.5; 514/252.12; 514/651

(57) ABSTRACT

Patients with aggressive breast cancer, including patients with estrogen receptor (ER) negative tumors and/or patients who have a disease-free interval from diagnosis to metastatic relapse of less than 36 months, have greater survival times than by treatment with DPPE and doxorubicin than patients receiving doxorubicin alone. Greater survival times are also achieved in patients receiving more than four cycles of treatment with DPPE and doxorubicin rather than doxorubicin alone. Greater survival times by treatment with DPPE and doxorubicin as compared to doxorubicin alone are further obtained in patients with metastatic breast cancer and who either have had no prior chemotherapy or no prior treatment type.

### FIELD OF THE INVENTION

The present invention relates to the treatment of breast cancer.

### BACKGROUND OF THE INVENTION

One of the major chemotherapeutic treatments is that of malignant growth (cancer) in humans. The objective of chemotherapy is the total extermination of clonogenic tumor or malignant cells, with minimal damage to the patient. However, one of the major limitations of the chemotherapeutic approach for managing human cancer is the general inability of anticancer drugs to discriminate between normal and tumorous cells. Anti-neoplastic agents have the lowest therapeutic indicies of any class of drugs used in humans and hence produce significant and potentially life-threatening toxicities. Certain commonly-used anti-neoplastic agents have unique and acute toxicities for specific tissues. For example, the vinca alkaloids possess significant toxicity for nervous tissues, while adriamycin has specific toxicity for heart tissue and bleomycin has for lung tissue. In general, almost all members of the major categories of anti-neoplastic agents have considerable toxicities for normal cells of gastrointestinal, epidermal and myelopoietic tissues.

Generally, the dose-limiting consideration for chemical management of cancer in humans is the toxicity that anti-neoplastic agents have for the pluripotent stem cells of myelopoietic tissue. This toxicity arises from the fact that most anticancer drugs function preferentially against proliferating cells but with no significant capacity to discriminate between cycling normal and cycling tumor tissues.

In U.S. Pat. Nos. 6,288,799, 5,859,065, 5,708,329, 5,747,543 and 5,618,846, all assigned to University of Manitoba and the disclosures of which are incorporated herein by reference, there is described an improved method for the in vivo chemotherapeutic treatment of cancer in which there is first administered a compound which inhibits normal cell proliferation while promoting malignant cell proliferation, specifically a potent antagonist selective for intracellular histamine receptors, in an amount sufficient to inhibit the binding of intracellular histamine to the receptors in normal and malignant cells. Following sufficient time to permit the inhibition of binding of intracellular histamine, a chemotherapeutic agent is administered. An enhanced toxic effect on the cancer cells from the chemotherapeutic agent is obtained while any adverse effect of the chemotherapeutic agent on normal cells, particularly bone marrow and gastrointestinal cells, is significantly ameliorated. One useful compound which inhibits normal cell proliferation while promoting malignant cell proliferation is N,N-diethyl-2-[4-(phenylmethyl)-phenoxy]ethanamine, abbreviated herein as DPPE.

### SUMMARY OF INVENTION

It has now surprisingly been found, in a Phase III clinical trial, that certain classes of patients with metastatic or recurrent breast cancer treated in accordance with the procedure described in the abovementioned patents using specific combinations of materials, exhibit greater survival than other patients. One class of patients in which enhanced survival was found were patients having estrogen receptor (ER). negative tumors. Another class of patients was those having a disease-free interval (DFI) from diagnosis to metastatic relapse of less than 36 months. ER negativity and short DFI are each generally accepted as surrogates for high grade malignancy, i.e. more aggressive disease. Other patients with high grade malignancy include patients with inflammatory breast cancer and T3 to T4 breast cancer requiring neoadjuvant treatment.

The endpoint of increased survival is rarely if ever achieved in any known active regimen in metastatic breast cancer. The usual finding is an improvement in early endpoints (response rate and/or time to progression, otherwise known as progression-free survival) but the improvement almost never translates into a significant increase in overall survival, as exhibited in patients treated in accordance with the present invention. Accordingly, it is a surprising result that the pretreatment with DPPE in patients with aggressive breast cancer increased overall survival by about 50% when compared to the control group.

Accordingly, in one aspect, the present invention provides a method of achieving enhanced survival in human patients with aggressive breast cancer, such as patients with estrogen receptor negative tumors and/or patients who have a disease-free interval from diagnosis to metastatic relapse of less than 36 months, which comprises:

(a) first administering to said patients at least one diphenyl compound of the formula: 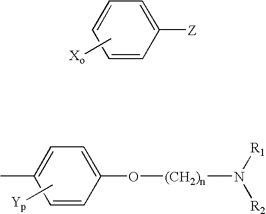
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient a chemotherapeutic agent active in breast cancer.

In the application of the present invention, the diphenyl compound and the chemotherapeutic agent are generally administered by intravenous infusion. In one preferred procedure, a solution of the diphenyl compound is administered to the patient over a desired period of time prior to administration of the chemotherapeutic agent and a solution of the chemotherapeutic agent in combination with the diphenyl compound then is administered for the period of administration of the chemotherapeutic agent. If desired, a solution of the diphenyl compound is administered after completion of the administration of the chemotherapeutic agent for a desired period of time to ameliorate side effects from the chemotherapeutic agent administration.

Breast cancer patients referred to as “estrogen receptor negative” (ER−), are those patients presenting with breast tumours the phenotype of which is determined to be substantially negative for estrogen receptor. Various established methods can be applied to make this determination. For instance, such phenotyping is typically performed on fresh or frozen biopsy tissue using an enzyme immunoassay in which antibody to the estrogen receptor is coupled to an enzyme, the presence of which is revealed by staining, and then detected either visually or using an imaging system. Results generally are expressed using a scoring system which may include the proportion of cells staining positive and the intensity of that staining relative to a control slide. Reference may be made, for instance, to the assay protocols reported by Elias et al in J. Cell. Biochem Suppl 19, 134-7, 1994, and Saccani-Jotti et al in J. Clin Pathol 47(10):900-5, 1994. Estrogen receptor negative (ER−) tumours are those which score significantly below a control sample positive for estrogen receptor.

Specific subjects for the present treatment are those patients presenting with breast cancer that is especially aggressive, as revealed by their disease-free interval (DFI). The term “disease-free interval” refers to the period extending from first histological diagnosis of breast cancer to diagnosis of metastatic/recurrent breast cancer, which in the case of aggressive metastatic/recurrent breast cancer is a period usually of less than about 36 months, for example; a period of 6 to 36 months or shorter.

It has further surprisingly been found, in the Phase III clinical trial, that the number of cycles of chemotherapeutic treatment of patients with metastatic or recurrent breast cancer by the procedure described in the above patents affects overall survival of patients in comparison to treatment with the chemotherapy agent alone. In this regard, it was found that patients receiving more than four cycles of DPPE and doxorubicin treatment exhibited a significant increase in overall survival as compared to patients treated with doxorubicin alone, whereas patients receiving four cycles or less did not.

Accordingly, in another aspect of the present invention, there is provided a method of achieving enhanced survival in human patients with metastatic breast cancer, which comprises:

subjecting patients to chemotherapy treatment for more than four cycles at predetermined intervals, each said cycle comprising:

(a) first administering to said patients at least one diphenyl compound of the formula: 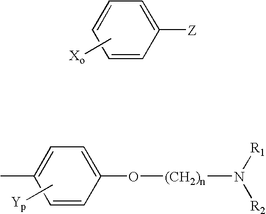
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient a chemotherapeutic agent active in breast cancer.

It has been additionally found, in the Phrase III clinical trial, that a significant increase in overall survival occurred in patients who had no previous chemotherapy or no previous treatment, including chemotherapy, radiotherapy and/or hormone treatment, in a comparison of DPPE/doxorubicin with doxorubicin alone.

Accordingly, a further aspect of the present invention provides a method of achieving enhanced survival in human patients with metastatic breast cancer, which comprises:

(a) selecting for chemotherapy treatment patients who have had no prior chemotherapy or any previous treatment type, and

(b) subjecting said selected patients to chemotherapy treatment for a plurality of cycles at predetermined intervals, each said cycle comprising:
(i) first administering to said selected patients at least one diphenyl compound of the formula: 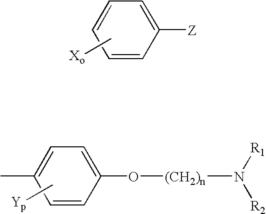
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and
(ii) following sufficient time to permit inhibition of binding of intracellular histamine, subsequently administering to the patient a chemotherapeutic agent active in breast cancer.

### BRIEF DESCRIPTION OF DRAWINGS

FIGS. 1A and 1B are graphical representations of results of a human Phase III clinical trial outlined below and depict the survival time of ER+ (FIG. 1A) and ER− (FIG. 1B) patients with metastatic and/or recurrent breast cancer treated with a combination of DPPE and doxorubicin in comparison with a control treatment with doxorubicin alone (solid line, DPPE/DOX; dotted line, DOX);

FIG. 2 is a graphical comparison of the survival time of patients with ER+ and ER− tumors treated with the DPPE/DOX combination (solid line, negative; dotted line, positive). Both groups of patients exhibited greater survival time in comparison with treatment with DOX alone, but patients with ER− tumors had longer survival than those with ER+ tumor;

FIGS. 3A to 3C are graphical representations of results of the human Phase III clinical trial outlined below and depict the survival by duration for patients with metastatic and/or recurrent breast cancer with DFIs of less than six months FIG. 3A), from greater than 6 months to 36 months (FIG. 3B) and greater than 36 months (FIG. 3C) with a combination of DPPE and doxorubicin in comparison with doxorubicin alone (solid line, DPPE, DOX; dotted line DOX);

FIGS. 4A and 4B are graphical representations of results of the human Phase III clinical trial outlined below and depict the survival time for patients with metastatic and/or recurrent breast cancer subjected to 4 or less cycles of chemotherapy (FIG. 4A) and more than 4 cycles of chemotherapy (FIG. 4B) with a combination of DPPE and doxorubicin in comparison with doxorubicin alone (solid line, DPPE/DOX; dotted line, DOX);

FIG. 5 is a graphical representation of results of the human Phase III clinical trial outlined below and depicts the survival by duration for patients with metastatic and/or recurrent breast cancer and who have had no prior chemotherapy treatment with a combination of DPPE/DOX in comparison with doxorubicin alone (solid line, DPPE/DOX; dotted line, DOX); and

FIG. 6 is a graphical representation of results of the human Phase III clinical trial outlined below and depicts the survival by duration for patients with metastatic and/or recurrent breast cancer and who had no previous treatment type with a combination of DPPE/DOX in comparison with doxorubicin alone (solid line, DPPE/DOX; dotted line, DOX).

### GENERAL DESCRIPTION OF INVENTION

In the present invention, a diphenyl compound is used which is a potent antagonist of histamine binding at the intracellular histamine receptor and is administered in an amount sufficient to inhibit the binding of intracellular histamine at the intracellular binding rate (H_{IC}) in normal cells. Such compounds exhibit a pK₁ of at least about 5, preferably at least about 5.5.

Specific potent compounds which are useful in the present invention are diphenyl compounds of the formula: 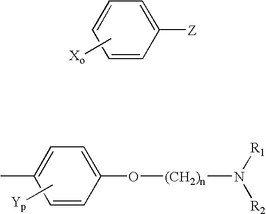
wherein X and Y are each fluorine, chlorine or bromine, Z is an alkylene group of 1 to 3 carbon atoms or ═C═O, o and p are 0 or 1, R₁ and R₂ are each alkyl groups containing 1 to 3 carbon atoms or are joined together to form a hetero-ring with the nitrogen atom and n is 1, 2 or 3. Pharmaceutically-acceptable salts of the diphenyl compounds may be employed.

Alternatively, the benzene rings may be joined to form a tricyclic ring, in accordance with the structure:

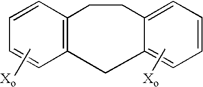

In one preferred embodiment, the group 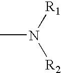
is a diethylamino group, although other alkylamino groups may be employed, such as dimethylamino, and, in another preferred embodiment, a morpholino group, although other heterocyclic ring groups may be employed, such as piperazino. o and p are usually 0 when Z is an alkylene group and o may be 2. In one particularly preferred embodiment, Z is —CH₂—, n is 2, o and p are each 0 and 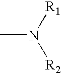
is a diethylamino group. This compound, namely N,N-diethyl-2-[4-(phenylmethyl)-phenoxy]ethanamine, which may be in the form of the free base or in the form of its hydrochloride salt, is abbreviated herein as DPPE. In addition to a methyl group linking the benzene rings, other linking groups may be employed, such as ═C═O. Other substitutents may be provided on the benzene rings in addition to the halogen atoms, for example, an imidazole group.

The diphenyl compound employed in the present invention is administered to the patient in any convenient manner, such as by intravenous injection of a solution thereof in an aqueous pharmaceutically-acceptable vehicle. The diphenyl compound is administered to the patient over a period of time before administration of the chemotherapeutic agent.

The chemotherapeutic agent employed herein is one which is active in breast cancer. Such chemotherapeutic agents active in breast cancer include anthracyclines, such as doxorubicin and epirubicin; anthracene diones, such as mitoxantrone; and taxanes, such as Taxol (a trademark of Bristol-Myers Squibb for paclitaxel) and Taxotere (a trademark of Aventis Pharma for docetaxel). The chemotherapeutic agent, or a mixture of such agents, is administered in any manner consistent with its normal manner of administration in conventional breast cancer therapy, often by intravenous infusion of a solution thereof. Specific combinations of chemotherapeutic agents which may be used in the procedures of the present invention include doxorubicin or epirubicin with Taxol or Taxotere.

The administration of the diphenyl compound to the patient prior to administration of the chemotherapeutic agent is necessary in order to permit the diphenyl compound to inhibit the binding of intracellular histamine in normal and malignant cells and thereby, in effect, shut down the proliferation of the normal cells, but increase proliferation of malignant cells.

The length of time prior to administration of the chemotherapeutic agent(s) that the diphenyl compound is administered depends on the diphenyl compound, its mode of administration and the size of the patient. Generally, the diphenyl compound is administered to the patient for about 30 to about 90 minutes, preferably about 60 minutes, prior to administration of the chemotherapeutic agent(s).

The quantity of diphenyl compound administered to the patient depends on the side effects to be ameliorated, but should be at least sufficient to inhibit binding of intracellular histamine in normal cells. The quantity required to achieve the beneficial effects of the present invention depends upon the diphenyl compound employed, the chemotherapeutic agent(s) employed and the quantity of such agent(s) employed.

In general, the quantity of diphenyl compound employed in humans is from, about 8 to about 320 mg/M² of human to which the diphenyl compound is administered, with about 8 and 240 mg/M² being the optimal dose for gastrointestinal and bone marrow protection, respectively. Over this dose range, the present invention is able to achieve an enhanced chemotherapeutic effect on breast cancer cells while, at the same time, also protecting normal cells from damage by the chemotherapeutic agent(s) in a wide variety of circumstances where traditional chemotherapy leads to damage of normal cells or tissues not involved in the disease process.

In the treatment of metastatic and/or recurrent breast cancer, the diphenyl compound preferably is used in an amount of about 3 to about 10 mg/kg of patient administered intravenously over a period of about 30 to about 90 minutes prior to administration of the chemotherapeutic agent(s) and continuing for the period of administration of the chemotherapy agent(s). In the specific Phase III clinical trial described herein, there was employed 5.3 mg/kg of DPPE in the form of the base (equivalent to 6 mg/kg of DPPE in the form of its hydrochloride), administered intravenously as an aqueous solution thereof over 80 minutes, with the last twenty minutes being accompanied by infusion of the specific chemotherapeutic agent.

The anthracycline chemotherapy agent active in breast cancer which is employed herein preferably is used in a total amount of about 50 to about 75 mg/M² of patient consistent with the identity of the anthracycline chemotherapy agent(s). In the specific Phase III clinical trial described herein, there was employed 60 mg/M² of doxorubicin administered over the last 20 minutes of infusion of the DPPE solution. However, epirubicin is equally potent and may be used in place of doxorubicin.

As set forth herein, a Phase III clinical trial was conducted on patients having metastatic and/or recurrent breast cancer in which one group of patients was administered DPPE followed by doxorubicin while a control group was administered doxorubicin alone. Various data from the clinical trial were collected and analyzed.

The present invention is based on the analysis of the survival times of the patients. In this regard, it was surprisingly found that, when patients with ER+ and ER− tumors were compared, while survival times of the DPPE/DOX treatment patients were longer than DOX treated patients for both patients with ER+ and ER− tumors, patients with ER− tumors were statistically longer survivors than patients with ER+ tumors.

In addition, it was surprisingly found that the combination of DPPE/DOX lead to greater survival times for patients having disease-free intervals of less than 36 months when compared to patients treated with DOX alone, whereas little difference was seen between the groups of patients with DFI's of greater than 36 months.

Since both ER− tumors and DFI's less than 36 months are generally accepted surrogates of aggressive breast cancer, this unexpected information forms the basis for a cancer treatment for aggressive breast cancer, which leads to enhanced survival. Other manifestations of aggressive breast cancer are inflammatory breast cancer and T3 to T4 breast cancer, which also can be treated by neoadjuvant therapy according to the invention.

This unexpected information also forms the basis of a diagnostic procedure which enables patients with metastatic and/or recurrent breast cancer to be tested to determine their expectation for survival (or time to death) treated with DPPE and anthracyclines by determining whether or not the tumors are ER negative and/or the patients have a DFI less than 36 months. Such test procedure forms another aspect of the present invention.

Accordingly, in another aspect of the present invention, there is provided a method useful in the treatment of a patient presenting with breast cancer to enhance the survival thereof, the method comprising:
(a) identifying patients with breast cancer having an estrogen receptor negative phenotype, and then
(b) subjecting those identified patients to a breast cancer treatment regimen in which a chemotherapy agent active in the treatment of breast cancer is administered following pretreatment of the patient with a diphenyl compound of the formula: 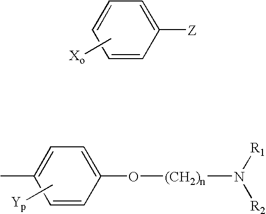
   wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof effective to potentiate the effect of the chemotherapy agent.

The patients may be further identified as having recurrent and/or metastatic breast cancer and a disease-free survivals of less than about 36 months.

In a further aspect of the present invention, there is provided a method useful in the treatment of a patient presenting with breast cancer to enhance the survival of the patient, the method comprising the steps of:
(a) identifying patients having recurrent and/or metastatic breast cancer and a disease-free interval of less than about 36 months, and then
(b) subjecting those identified patients to a breast cancer treatment regimen in which a chemotherapeutic agent active in the treatment of breast cancer is administered following pretreatment of the patient with an amount of a diphenyl compound of the formula: 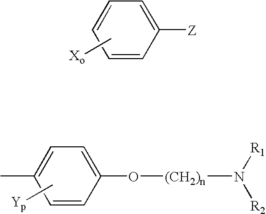
   wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof effective to potentiate the effect of the chemotherapy agent.

The patients may be further identified as having breast cancer of the estrogen receptor negative phenotype.

In these procedures, the diphenyl compound, the chemotherapy agent and the breast cancer treatment regimen may include the options referred to above. The diphenyl compound employed is preferably DPPE, in the free base form or in the form of the hydrochloride, and the chemotherapy agent in preferably doxorubicin or epirubicin.

Further analysis of the data showed that a significant increase in survival was dependent on the number of cycles of DPPE/doxorubicin administered in comparison to doxorubicin alone. Patients receiving more than 4 cycles of DPPE/doxorubicin treatment had a significant increase in overall survival in comparison to doxorubicin alone, whereas patient receiving 4 or less cycles did not.

Additional analysis of the data showed that the most significant increase in median overall survival in patients receiving DPPE/doxorubicin treatment in comparison to doxorubicin alone occurred in patients who with metastatic disease had no previous chemotherapy or no previous treatment type.

In procedures carried out on said latter patients, the diphenyl compound, the chemotherapy agent and the breast cancer treatment regimen may include the options referred to above. The diphenyl compound is prefer ably DPPE, in the form of the free base or in the form of the hydrochloride, and the chemotherapy agent is preferably doxorubicin or epirubicine.

### EXAMPLE

### Example 1

This Example describes a Phase III clinical trial of the treatment of patients and metastatic and/or recurrent breast cancer.

Patients were treated with doxorubicin (DOX) alone or a combination of doxorubicin and DPPE. DPPE in the free base form, was administered intravenously at a dose of 5.3 mg/kg over 80 minutes with doxorubicin administered at a dose of 60 mg/M² over the last 20 minutes while the control group received a dose of 60 mg/M² of doxorubicin alone. The patients were subjected to a number of cycles of chemotherapy, each followed by a 21 to 28 day rest period, until a cumulative dose of up to 450 mg/M² of doxorubicin had been administered to the patient.

305 patients participated in the study. 152 patients were randomized to DPPE/doxorubicin and 153 patients received doxorubicin alone. Median age was 53 years, 90% had received no prior chemotherapy for metastatic disease and 60% had viceral disease.

### Example 2

This Example analyzes the data obtained in the Phase III clinical trial described in Example 1.

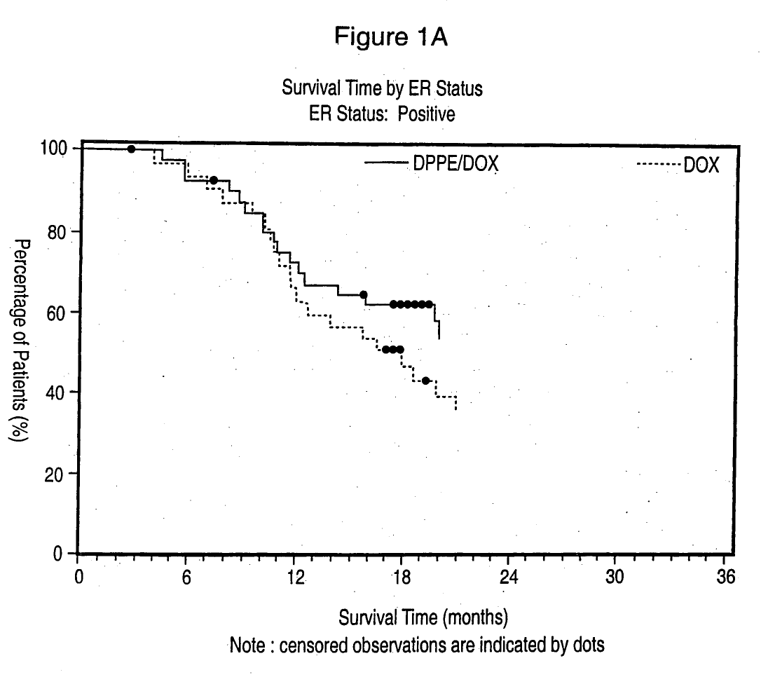

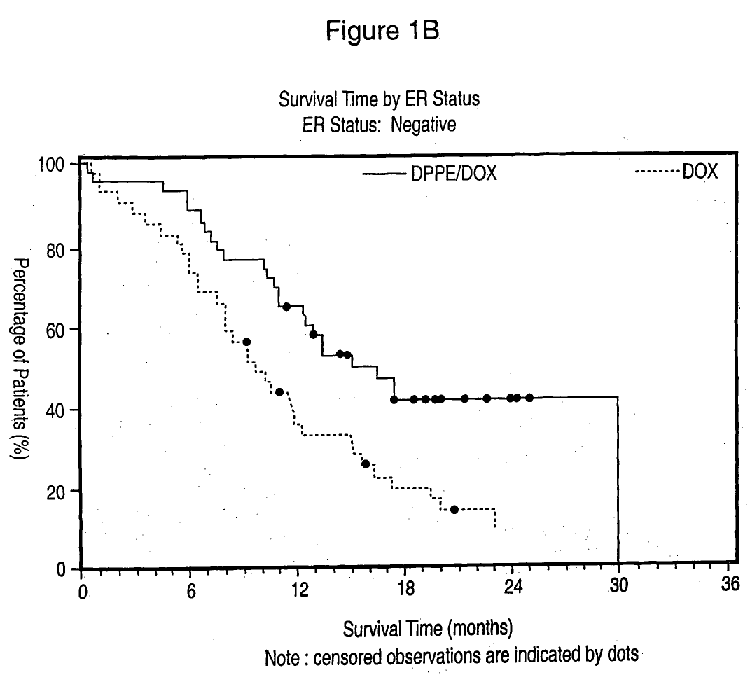

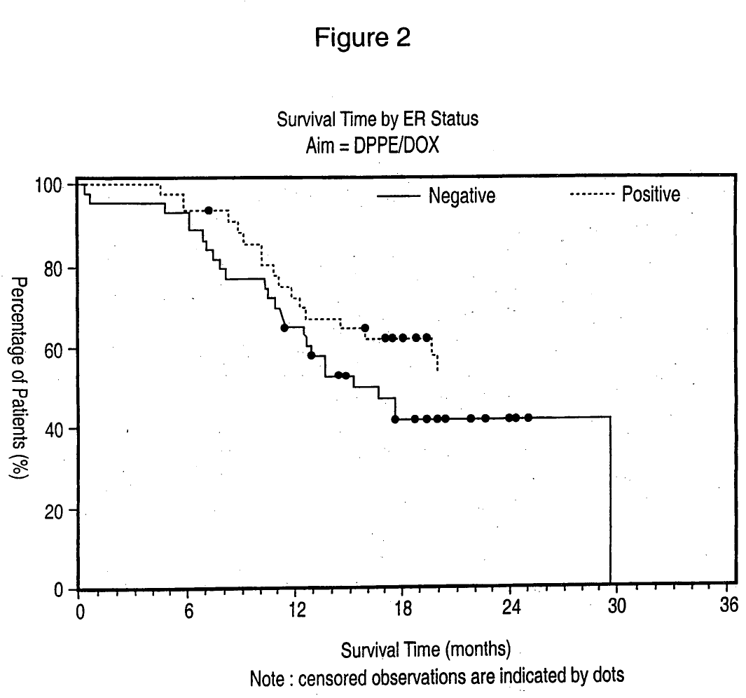

The survival time of patients was determined in relation to ER status for patients receiving the DPPE/DOX combination and DOX alone. These results are shown in FIGS. 1A (ER positive) and 1B (ER negative). In addition, the survival time for the DPPE/DOX combination patients was compared for the ER positive and negative groups. These results are shown in FIG. 2.

As can be seen from these results, not only is the combination of DPPE/DOX more effective in terms of patient survival than DOX alone for patients both with ER positive and ER negative tumors (FIGS. 1A and 1B) but the combination DPPE/DOX treatment led to greater survival for patients with tumors which were ER negative in comparison with those which were ER positive (FIG. 2).

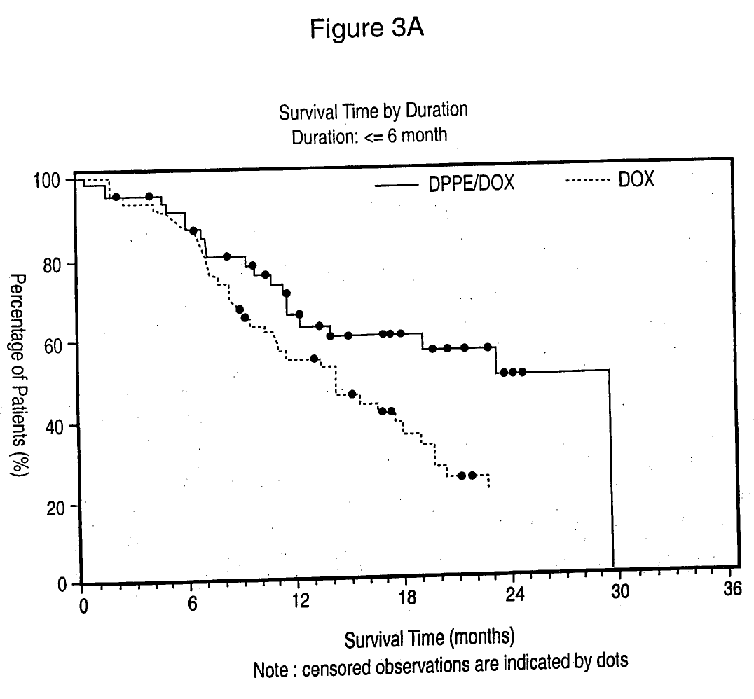

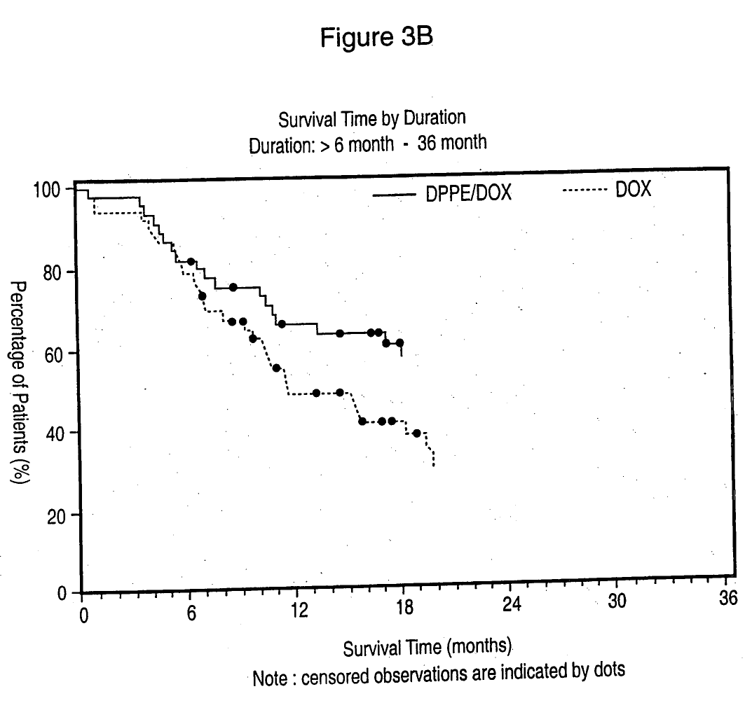

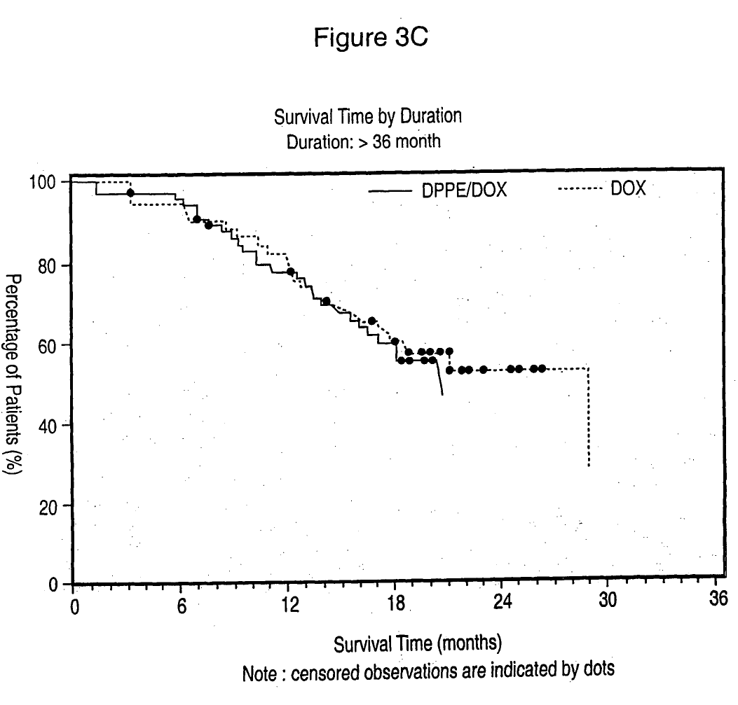

In addition, the survival times of patients was determined in relation to disease-free intervals for patients receiving the DPPE/DOX combination and DOX alone. These results are shown in FIGS. 3A (DFI less than 6 months), 3B (DFI from 6 to 36 months), and 3C (DFI greater than 36 months).

As can be seen from these results, the combination of DPPE/DOX was more effective in terms of patient survival than DOX alone for patients with DFI's of less than 36 months.

### Example 3

This Example further analyzes the data obtained in the Phase III clinical trial described in Example 1.

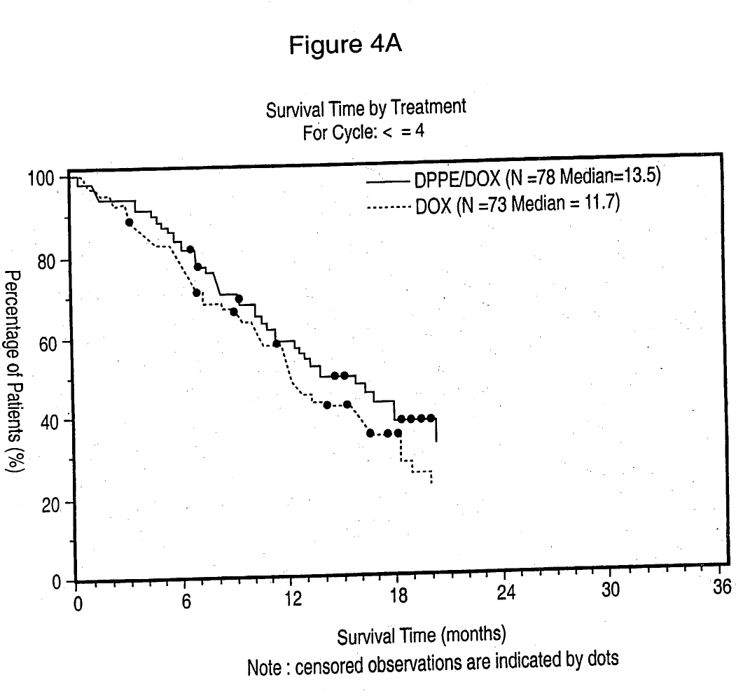

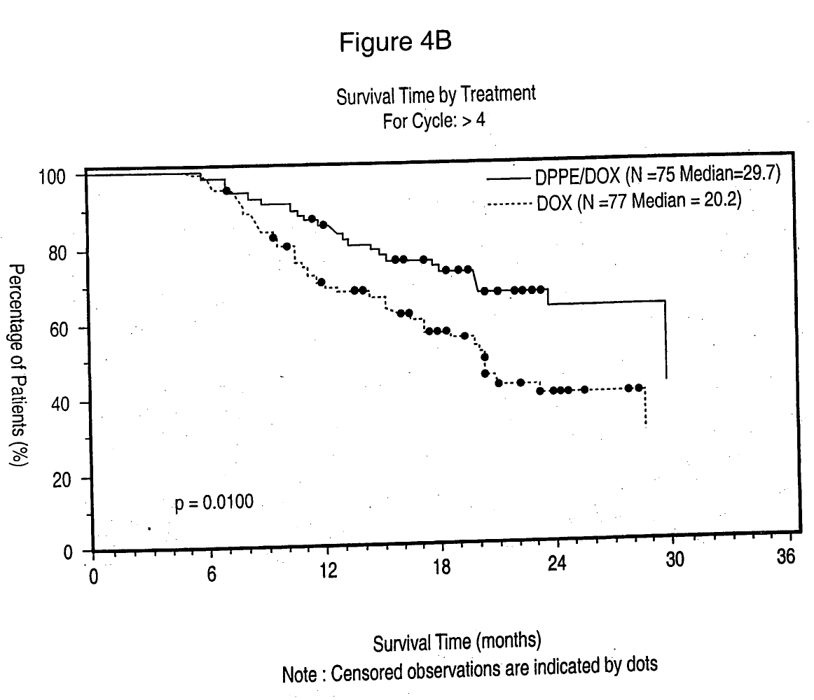

The survival times of patients was determined in relation to the number of cycles of treatment for patients receiving the DPPE/DOX combination and DOX alone. These results are shown in FIG. 4A for patients receiving 4 or less cycles of chemotherapy treatment and FIG. 4B for patients receiving more than 4 cycles of chemotherapy treatment.

As may be seen from these results, patients receiving more than four cycles of chemotherapy treatment by DPPE/DOX exhibited a significant increase in overall survival, as compared to those receiving four or less cycles.

The survival time for patients also was determined for those patients with metastatic breast cancer receiving no prior chemotherapy treatment in relation to those also had received prior chemotherapy treatment and for patients that had received no previous treatment type (i.e. no prior chemotherapy, radiotherapy and/or hormone treatment). The survival times were determined for patients receiving the DPPE/DOX combination and DOX alone.

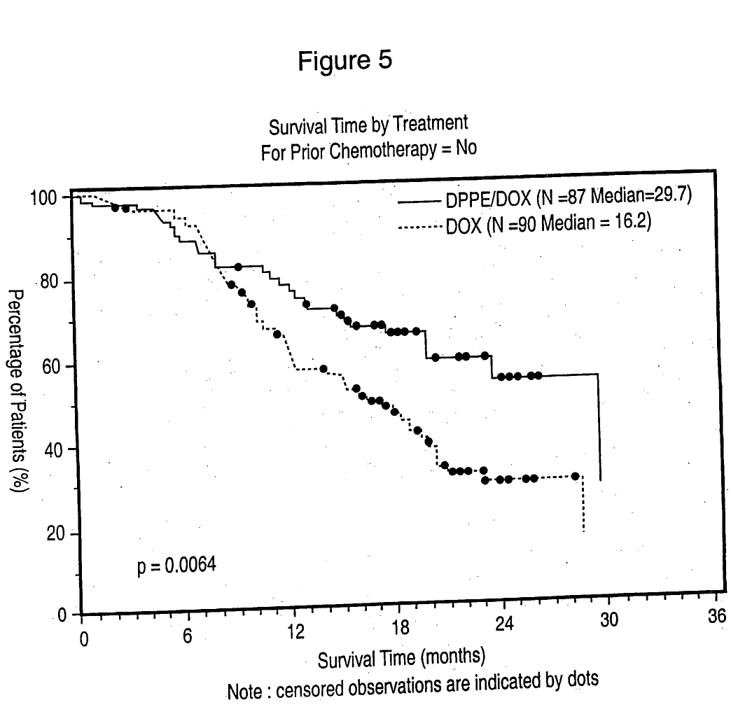

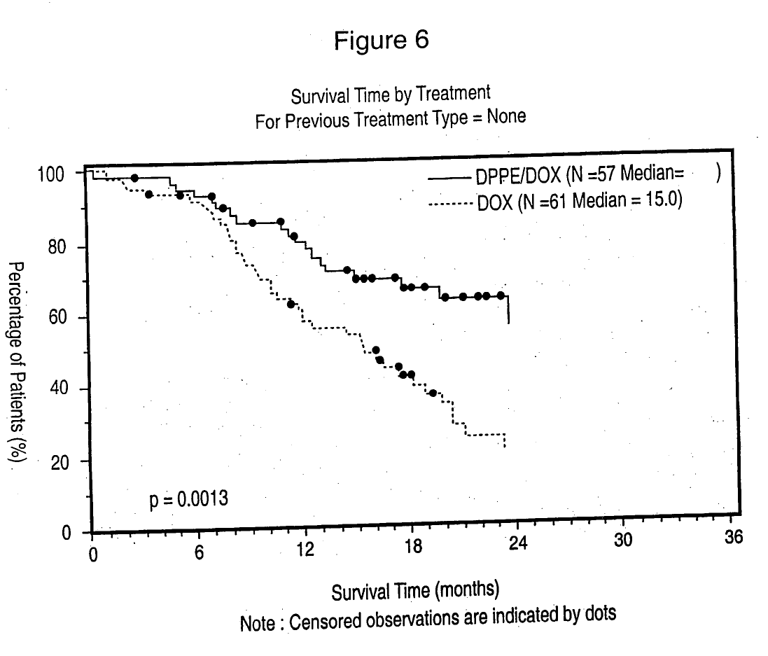

These results are shown in FIGS. 5 (no prior chemotherapy) and 6 (no previous treatment type). As may be seen in these Figures, a significant increase in median overall survival as between treatment with the DPPE/DOX combination and DOX alone was observed in patients with metastatic disease who had no previous chemotherapy namely 29.7 months (N=87) for the DPPE/DOX combination as comparison to 16.2 months N=90) (P=0.006) for DOX alone, or no previous treatment, namely greater than 24 months (N=57) for the DPPE/DOX combination in comparisor to 15 months (N=60) (P=0.001) for DOX alone.

### SUMMARY OF INVENTION

In summary of this invention, the present invention provides a method o achieving enhanced survival for patients with metastatic and/or recurrent breast cancer Modifications are possible within the scope of the invention.

1. A method of achieving enhanced survival in human patients with aggressive metastatic breast cancer, which comprises:
(a) first administering to said patients at least one diphenyl compound of the formula: 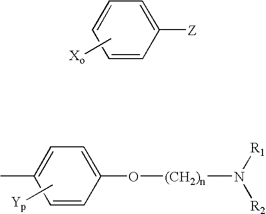
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and
(b) following sufficient time to permit inhibition of binding of intracellular histamine, subsequently administering to the patient a chemotherapeutic agent active in breast cancer.

2. The method of claim 1 wherein the group 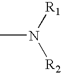
is a diethylamino group, a dimethylamino group, a morpholino group or a piperazino group.

3. The method of claim 1 wherein the group 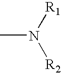
is a diethylamino group, Z is —CH₂, n is 2 and o and p are each 0.

4. The method of claim 3 wherein diphenyl compound is in the form of a hydrochloride salt or free base.

5. The method of claim 1 wherein said chemotherapeutic agent active in breast cancer is doxorubicin.

6. The method of claim 4 wherein said chemotherapeutic agent specific for breast cancer is doxorubicin or epirubicin.

7. The method of claim 1 wherein said diphenyl compound is administered to the patient about 30 to about 90 minutes prior to said administration of said chemotherapeutic agent.

8. The method of claim 7 wherein said time is about 60 minutes.

9. The method of claim 6 wherein said diphenyl compound is administered by intravenous infusion of a solution thereof over a period of time of up to about 90 minutes prior to administration of said chemotherapeutic agent and is maintained during administration of said chemotherapeutic agent.

10. The method of claim 9 wherein said diphenyl compound is administered for about 60 minutes prior to administration of said chemotherapeutic agent and is maintained during about 20 minutes intravenous infusion of said chemotherapeutic agent.

11. The method of claim 7 wherein said diphenyl compound is administered in an amount of about 8 to about 240 mg/M² of said patient.

12. The method of claim 11 wherein said amount is about 3 to about 10 mg/kg of patient.

13. The method of claim 9 wherein said diphenyl compound is administered in an amount of about 3 to about 10 mg/kg of patient.

14. The method of claim 10 wherein said diphenyl compound is administered in an amount of about 6 mg/kg in the form of the hydrochloride salt or 5.3 mg/kg in the form of the free base.

15. The method of claim 11 wherein said chemotherapeutic agent is administered in an amount of about 50 to about 75 mg/M² of patient.

16. The method of claim 14 wherein said chemotherapeutic agent is administered in an amount of about 60 mg/M² of patient.

17. The method of claim 1 wherein said patients with aggressive breast cancer are patients who have estrogen receptor negative tumors.

18. The method of claim 1 wherein said patients with aggressive breast cancer are patients who have a disease-free interval from diagnosis to metastatic relapse of less than 36 months.

19. A method useful in the treatment of a patient presenting with breast cancer to enhance the survival of the patient, the method comprising:
(a) identifying patients with breast cancer having an estrogen receptor negative phenotype, and then
(b) subjecting those identified patients to a breast cancer treatment regimen in which a chemotherapy agent active in the treatment of breast cancer is administered following pretreatment of the patient with an amount of a diphenyl compound having the formula: 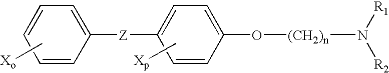
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof effective to potentiate the effect of the chemotherapeutic agent.

20. The method of claim 19 wherein the patient is further identified having recurrent and/or metastatic breast cancer and a disease-free interval of less than about 36 months.

21. A method useful in the treatment of a patient presenting with breast cancer to enhance the survival of the patient comprises the steps of:
(a) identifying patients having recurrent and/or metastatic breast cancer and a disease-free interval of less than about 36 months, and then
(b) subjecting those identified patients to a breast cancer treatment regimen in which a chemotherapeutic agent active in the treatment of breast cancer is administered following pretreatment of the patient with an amount of diphenyl compound of the formula: 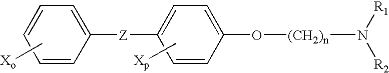
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof effective to potentiate the effect of the chemotherapy agent.

22. The method of claim 21 wherein the patient is further identified as having breast cancer of the estrogen receptor negative phenotype.

23. The method of claim 19 wherein the group 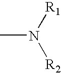
is a diethylamino group, Z is —CH₂, n is 2 and o and p are 0.

24. The method of claim 23 wherein said diphenyl compound is used in the form of its hydrochloride salt or free base.

25. The method of claim 24 wherein chemotherapy agent is doxorubicin or epirubicin.

26. The method of claim 24 wherein said diphenyl compound is administered to the patient by intravenous infusion of a solution thereof over a period of up to about 90 minutes prior to administration of said chemotherapy agent and is maintained during administration of said chemotherapy agent.

27. A method of achieving enhanced survival in human patients with metastatic breast cancer, which comprises:
(a) selecting for chemotherapy treatment patients who have had no prior chemotherapy treatment or any previous treatment, and
(b) subject said selected patients to chemotherapy treatment for a plurality of cycles at predetermined intervals, each said cycle comprising:
(i) first administering to said selected patients at least one diphenyl compound of the formula: 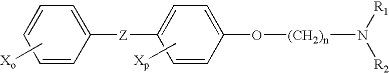
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and
(ii) following sufficient time to permit inhibition of binding of intracellular histamine, subsequently administering to the patient a chemotherapeutic agent active in breast cancer.

28. A method of achieving enhanced survival in human patients with metastatic breast cancer, which comprises:
subjecting patients to chemotherapy treatment for at least for cycles at predetermined intervals, each said cycle comprising:
(a) first administering to said patients at least one diphenyl compound of the formula: 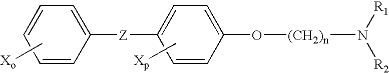
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each group containing 1 to 3 carbon atoms or are joined together to form a heterocyclic ring with the nitrogen atom and n is 1, 2 or 3, or pharmaceutically-acceptable salts thereof, and
(b) following insufficient time to permit inhibition of binding of intracellular histamine, subsequently administering to the patient a chemotherapeutic agent active in breast cancer.

29. (canceled)
